(19) United States
(12) Patent Application Publication
(10) Pub. No.: US 2019/0247533 A1
(43) Pub. Date: Aug. 15, 2019

(54) SCENT DIFFUSING SYSTEM AND DEVICE ELECTRICALLY CHARGED IN WIRELESS MANNER

(71) Applicant: DONGGUAN YIH TEH ELECTRIC PRODUCTS CO., LTD., Dongguan City (CN)

(72) Inventor: Ming Jen HSIAO, Miaoli County (TW)

(21) Appl. No.: 16/259,654

(22) Filed: Jan. 28, 2019

Related U.S. Application Data
(63) Continuation-in-part of application No. 15/893,962, filed on Feb. 12, 2018.
Application No. 15/893,962 is a continuation-in-part of application No. 16/157,994, filed on Oct. 11, 2018.

(51) Int. Cl. A61L 9/12; H02J 7/02

(52) U.S. Cl. CPC A61L 9/122; A61L 2209/133; H02J 7/025

(57) ABSTRACT

A scent diffusing system electrically charged in a wireless manner contains: a wireless charger and an aroma diffuser. The aroma diffuser includes a case, an air guide holder, a wireless charging receiver, and an air conveyor. The case has an open segment and a receiving chamber defined in the open segment. The air conveyor is accommodated in a bottom of the air guide holder, and the air guide holder is connected above the open segment of the case. The wireless charging receiver is accommodated in the receiving chamber, and the air conveyor is electrically connected with the wireless charging receiver. The aromatic element is received in the air guide holder of the aroma diffuser.

### BACKGROUND OF THE INVENTION

This application is a Continuation-in-Part of application Ser. No. 15/893,962, filed Feb. 12, 2018 and Ser. No. 16/157,994, filed Oct. 11, 2018.

### FIELD OF THE INVENTION

The present invention relates to a scent diffusing system and device which are electrically charged in a wireless manner.

### DESCRIPTION OF THE PRIOR ART

A conventional scent diffusing device is electrically connected with a power source by using an electrical plug, but it is easy to cause electric shock. The scent diffusing device or an electrical wire is connected with a heater of the scent diffusing device so that the heater heats an aromatic element. After running out the aromatic element, it is refilled or replaced after a removal of the electrical plug, thus having inconvenient operation.

The present invention has arisen to mitigate and/or obviate the afore-described disadvantages.

### SUMMARY OF THE INVENTION

The primary aspect of the present invention is to provide scent diffusing system and device which are electrically charged in a wireless manner and transmit energy by using a magnetic field so as to convert into electric energy to electrically charge an aroma diffuser easily and safely.

Another aspect of the present invention is to provide scent diffusing system and device which contain a wireless charger including multiple transmitting coil modules so as to electrically charge an aroma diffuser and/or various mobile devices or various terminal devices.

To obtain the above aspects, a scent diffusing system electrically charged in a wireless manner provided by the present invention contain: a wireless charger and an aroma diffuser.

The aroma diffuser includes a case, an air guide holder, a wireless charging receiver, and an air conveyor.

The case has an open segment and a receiving chamber defined in the open segment. The air conveyor is accommodated in a bottom of the air guide holder, and the air guide holder is connected above the open segment of the case. The wireless charging receiver is accommodated in the receiving chamber, and the air conveyor is electrically connected with the wireless charging receiver.

The aromatic element is received in the air guide holder of the aroma diffuser.

A scent diffusing device electrically charged in a wireless manner contains: an aroma diffuser including a case, an air guide holder, a wireless charging receiver, and an air conveyor.

The case has an open segment and a receiving chamber defined in the open segment, the air conveyor is accommodated in a bottom of the air guide holder, and the air guide holder is connected above the open segment of the case, the wireless charging receiver is accommodated in the receiving chamber, and the air conveyor is electrically connected with the wireless charging receiver.

The air guide holder has an opening, a first groove, a second groove, a guiding cavity, and a third groove, wherein the opening is defined on an upper end of the air guide holder, and the guiding cavity is defined between two inner walls of the air guide holder, the first groove extends to the air guide holder from the opening and communicates with an upper end of the guiding cavity, and the second groove is defined in a lower end of the guiding cavity, wherein an orifice of the second groove communicates with a bottom of the first groove; the guiding cavity has a first aperture formed on the upper end thereof and communicating with the first grove, the guiding cavity further has a second aperture formed on the lower end thereof and communicating with the receiving chamber; and the air conveyor is fixed in the third groove.

### BRIEF DESCRIPTION OF THE DRAWINGS

FIG. 1 is a cross sectional view showing the assembly of a scent diffusing system and device electrically charged in a wireless manner according to a preferred embodiment of the present invention.

FIG. 2 is a perspective view showing the assembly of a wireless charger of the system and device according to the preferred embodiment of the present invention.

FIG. 3 is a perspective view showing the exploded components of the wireless charger of the system and device according to the preferred embodiment of the present invention.

FIG. 4 is a perspective view showing the assembly of an aroma diffuser of the system and device according to the preferred embodiment of the present invention.

FIG. 5 is a perspective view showing the exploded components of the aroma diffuser of the system and device according to the preferred embodiment of the present invention.

FIG. 6 is a cross sectional view showing the assembly of a system and device according to another preferred embodiment of the present invention.

FIG. 7 is a perspective view showing the exploded components of an aroma diffuser of the system and device according to another preferred embodiment of the present invention.

FIG. 8 is a cross sectional view showing the assembly of a system and device according to another preferred embodiment of the present invention.

FIG. 9 is a cross sectional view showing the assembly of a system and device according to another preferred embodiment of the present invention.

FIG. 10 is a block diagram showing the assembly of the system and device according to the embodiments of FIGS. 1 and 9.

FIG. 11 is a cross sectional view showing the assembly of a system and device according to another preferred embodiment of the present invention.

FIG. 12 is a cross sectional view showing the assembly of a system and device according to another preferred embodiment of the present invention.

FIG. 13 is a block diagram showing the assembly of the system and device according to the embodiment of FIGS. 11 and 12.

### DETAILED DESCRIPTION OF THE PREFERRED EMBODIMENTS

Scent diffusing system and device electrically charged in a wireless manner according to a preferred embodiment of the present invention comprise: an aromatic element 5, wherein the aromatic element 5 is a scent bottle or a scent carrier in which aromatic fluid or aromatic substance is absorbed, wherein the aromatic fluid or the aromatic substance is selected from one of aromatic oil, essential oil, scented wax, essence, aromatic liquid, flower essence, aromatic agent, and solid or fluid which diffuses scent.

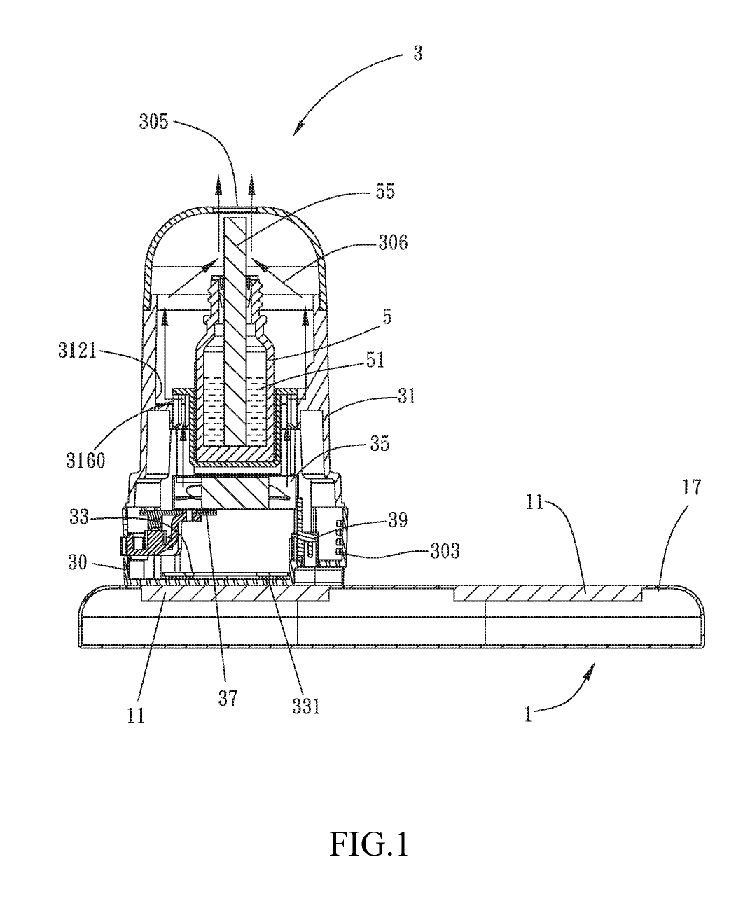

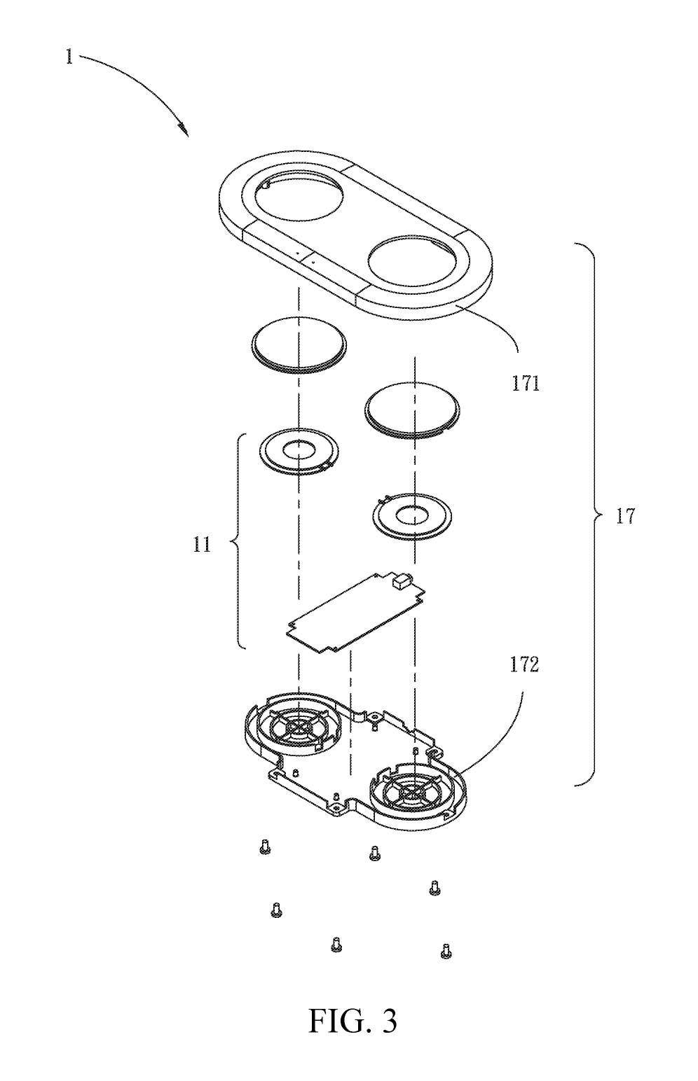

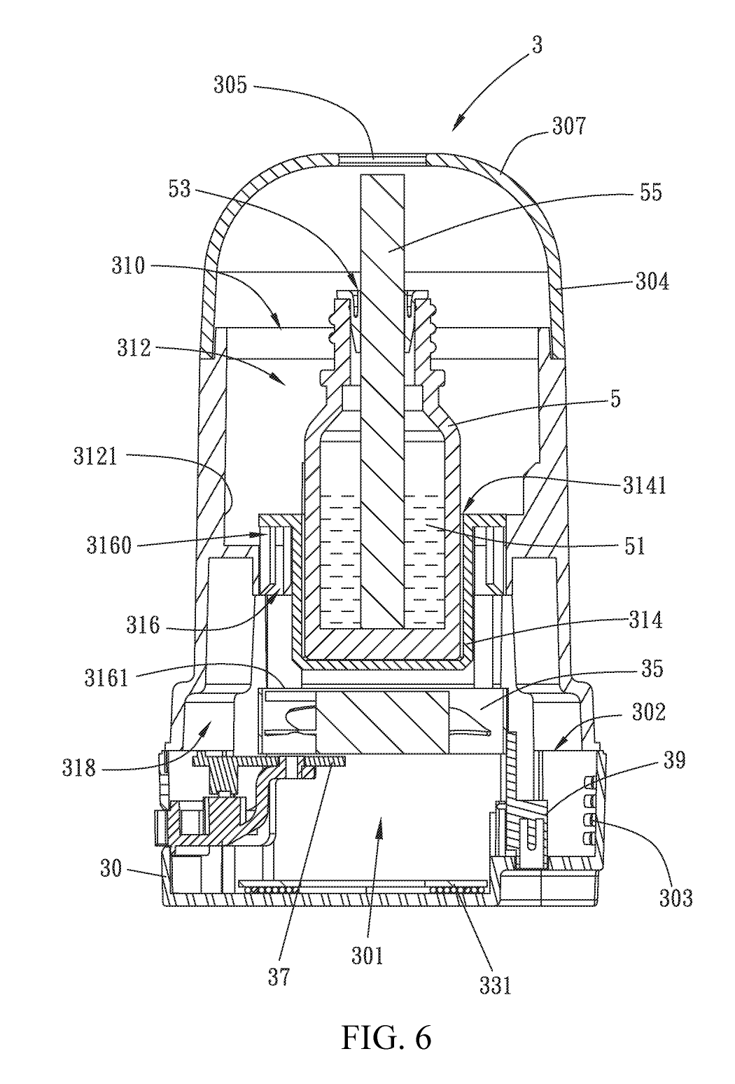

With reference to FIGS. 1-6, the scent diffusing system electrically charged in the wireless manner according to the preferred embodiment of the present invention further comprises: a wireless charger 1 and an aroma diffuser 3.

The aroma diffuser 3 includes a case 30, an air guide holder 31, a wireless charging receiver 33, and an air conveyor 35.

The case 30 has an open segment 302 and a receiving chamber 301 defined in the open segment 302. The air conveyor 35 is accommodated in a bottom of the air guide holder 31, and the air guide holder 31 is connected above the open segment 302 of the case 30. The wireless charging receiver 33 is accommodated in the receiving chamber 301, and the air conveyor 35 is electrically connected with the wireless charging receiver 33. The aromatic element 5 is received in the air guide holder 31 of the aroma diffuser 3.

The wireless charger 1 is electrically connected with a power source so that electricity is inputted to the wireless charger 1 from the power source to produce an electromagnetic signal, thus transmitting energy by using a magnetic field of the wireless charger 1. The wireless charging receiver 33 receives the electromagnetic signal and converts the electromagnetic signal into electric currents for driving the air conveyor 35 to transport external air out of an opening 310 via the air guide holder 31, a first groove 312, and the aromatic element 5, thus diffusing the scent.

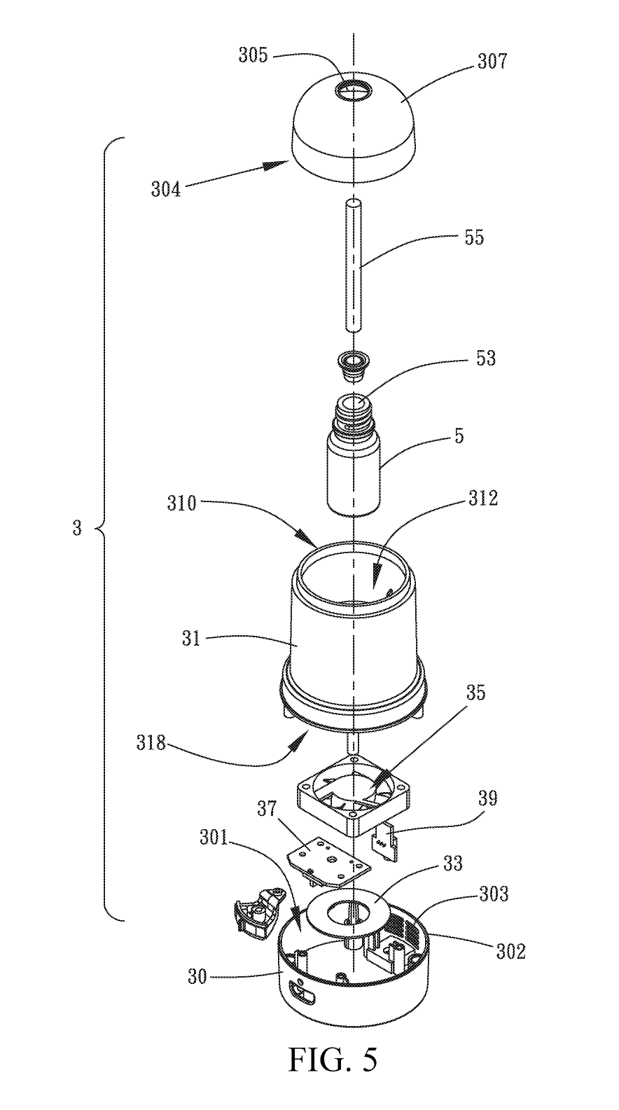

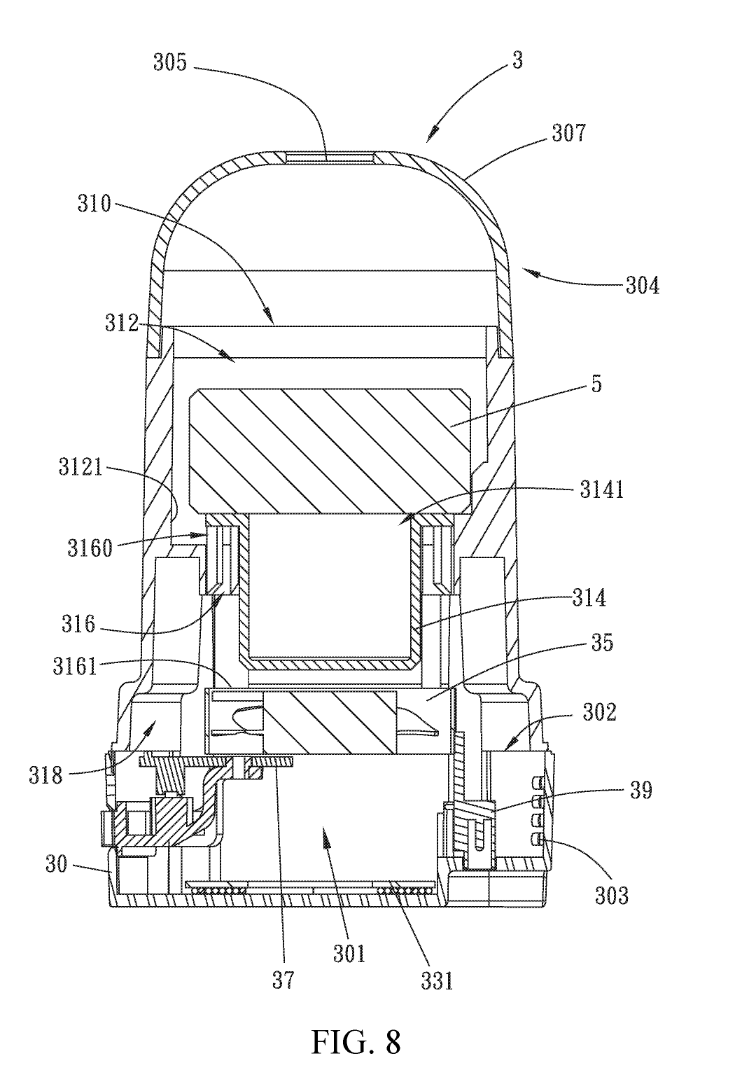

Referring to FIGS. 5-8, the air guide holder 31 has the opening 310, the first groove 312, a second groove 314, a guiding cavity 316, and a third groove 318. The opening 310 is defined on an upper end of the air guide holder 31, and the guiding cavity 316 is defined between two inner walls of the air guide holder 31. The first groove 312 extends to the air guide holder 31 from the opening 310 and communicates with an upper end of the guiding cavity 316, and the second groove 314 is defined in a lower end of the guiding cavity 316, wherein an orifice 3141 of the second groove 314 communicates with a bottom of the first groove 312. The guiding cavity 316 has a first aperture 3160 formed on the upper end thereof and communicating with the first grove 312. The guiding cavity 316 further has a second aperture 3161 formed on the lower end thereof and communicating with the receiving chamber 301. The air conveyor 35 is fixed in the third groove 318.

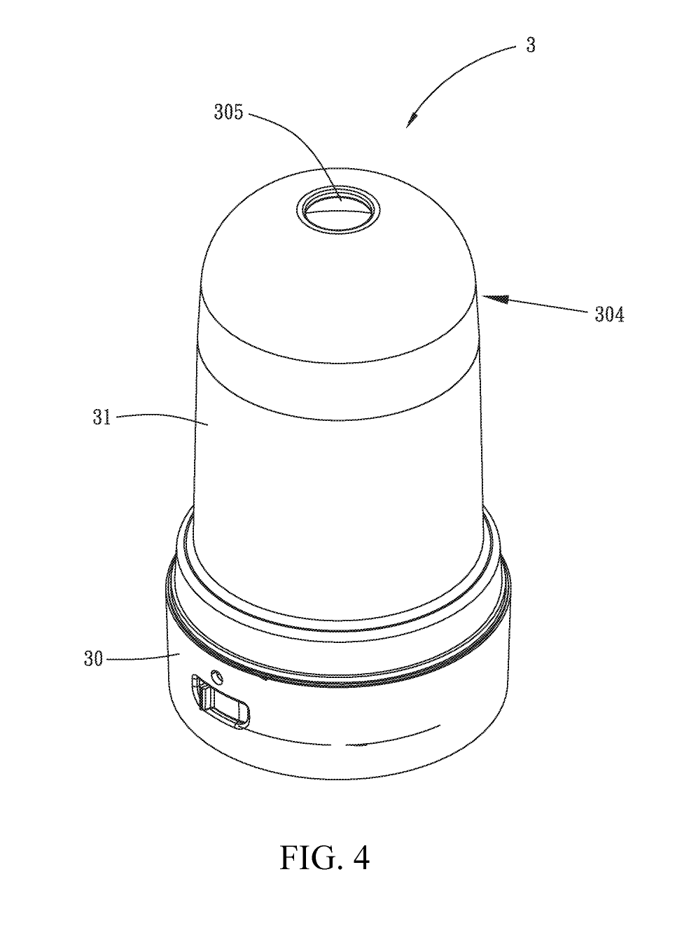

As shown in FIGS. 4-8, the receiving chamber 301 is not a closed space so that the air conveyor 35 draws and conveyors the external air out of the opening 310 from the second aperture 3161 via the guiding cavity 316, the first aperture 3160, and the first groove 312. In another embodiment, the case 30 further has an air inlet 303 formed on a side thereof and configured to draw the external air by using the air conveyor 35.

As illustrated in FIGS. 1, 5 and 6, the air conveyor 35 is any one of a fan, a pump, and a pressure bottle. Preferably, the air conveyor 35 is the fan so as to deliver the external air smoothly.

With reference to FIGS. 1, 5, and 6, the aromatic element 5 is the scent bottle in which the aromatic liquid 51 is accommodated. The scent bottle 5 has a mouth 53 and a diffusing element 55, wherein a first end of the diffusing element 55 is inserted into the scent bottle 5 to contact with and to absorb the aromatic liquid 51 toward the diffusing element 55, and a second end of the diffusing element 55 diffuses aroma molecules of the aromatic liquid 51 toward an external environment.

A bottom of the scent bottle 5 is accommodated in the second groove 314, the mouth 53 is located beside the first groove 312, a gap is formed among the scent bottle 5, the first groove 312, and the first aperture 3160 so that the external air flows in the gap from the air conveyor 35 (as denoted by an arrow of FIG. 1). The scent bottle 5 is accommodated into or is removed from the second groove 314 easily.

The wireless charging receiver 33 receives (i.e., induces) electromagnetic waves transmitted by the wireless charger 1 so as to transform the electromagnetic waves into the electricity for driving the air conveyor 35. The air conveyor 35 delivers the external air toward the diffusing element 55 via the second aperture 3161, the guiding cavity 316, the first aperture 3160, the first groove 312, and the opening 310, wherein at least one part of the diffusing element 55 is located in a scatter path 306, and the diffusing element 55 absorbs the aromatic liquid 51 and spreads the aroma molecules of the aromatic liquid 51, thus diffusing the scent toward the external environment.

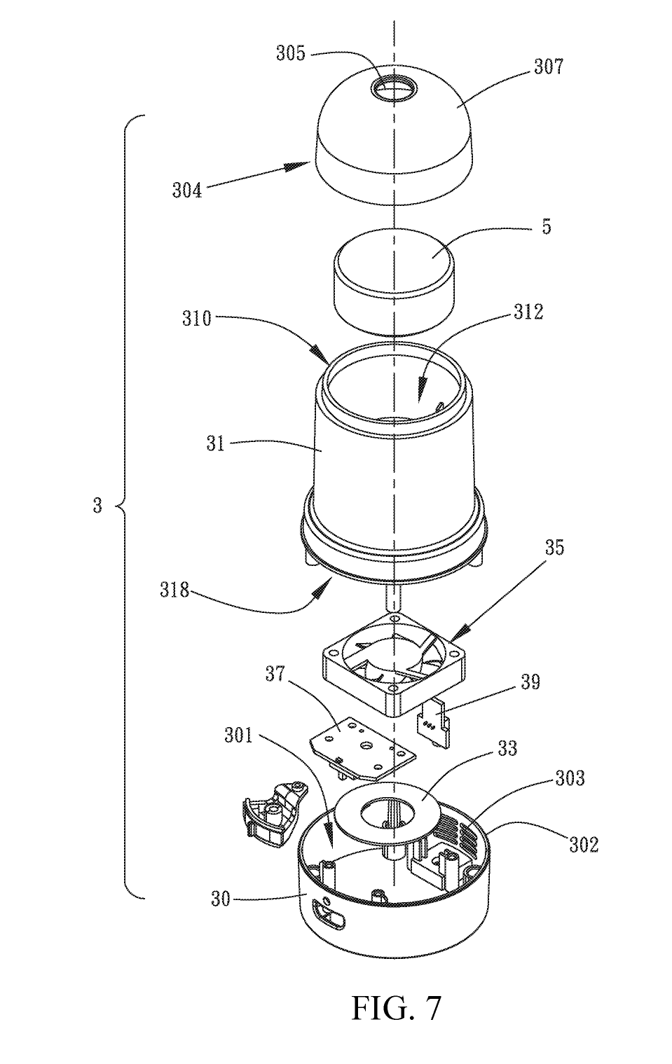

Referring to FIGS. 7-9, in another embodiment, the aromatic element 5 is the scent carrier which absorbs the aromatic liquid or the aromatic agent. The scent carrier 5 is porous and is made of any one of ceramic, plastic, plaster, foam, wood, fabric, and fiber, thus diffusing the aroma molecules of the aromatic liquid 51 toward the external environment.

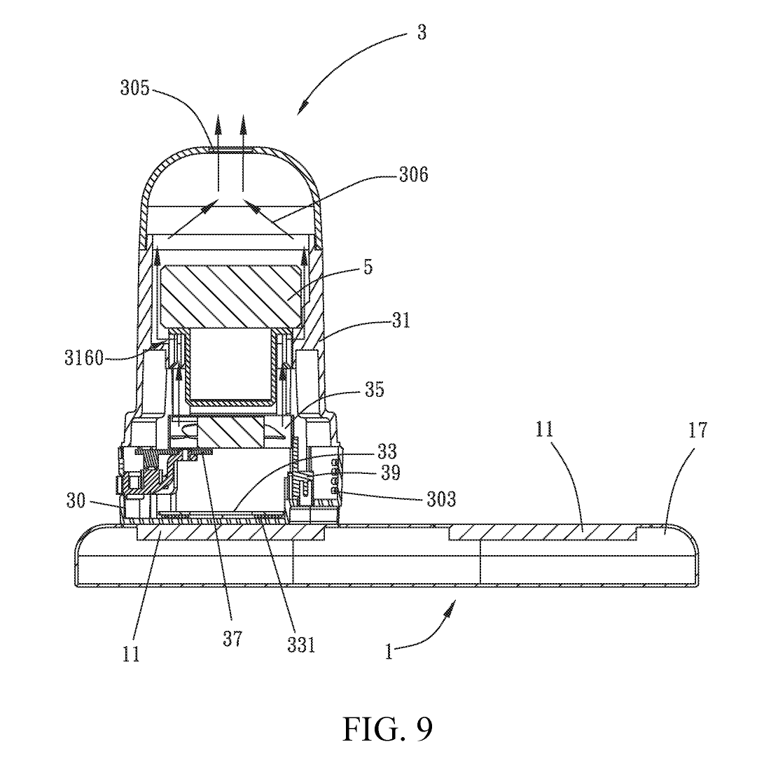

The scent carrier 5 is accommodated into the second groove 314 from the opening 310, and the gap is formed among the scent bottle 5, the first groove 312, and the first aperture 3160 so that the external air flows in the gap from the air conveyor 35 (as denoted by an arrow of FIG. 9). The scent carrier 5 is accommodated into or is removed from the second groove 314 easily. The air conveyor 35 delivers the external air toward the scent carrier 5 via the second aperture 3161, the guiding cavity 316, the first aperture 3160, the first groove 312, and the opening 310, wherein the scent carrier 5 is located in a scatter path 306, and the scent carrier 5 absorbs and diffuses aroma molecules of the scent carrier 5 toward the external environment.

Accordingly, the aroma diffuser 3 is not heated by a heater and diffuses the scent safely by ways of the air guide holder 31 and the air conveyor 35. The scent bottle 5 is removed from the second groove 314 easily so as to replace another scent bottle 5, then another bottle 5 is accommodated into the second groove 314 easily and quickly.

Preferably, the wireless charger 1 produces the magnetic field to send the energy, and the wireless charging receiver 33 of the aroma diffuser 3 is close to the electromagnetic signals (i.e., the magmatic field) of the wireless charger 1 so as to receive (i.e., induce) the electromagnetic signals and to transform the electromagnetic signals into the electric currents, hence the air conveyor 35 delivers the external air toward the aromatic element 5, and the aromatic element 5 diffuses the scent. Preferably, aromatic element 5 is accommodated into the aroma diffuser 3, and the aroma diffuser 3 is mounted on the wireless charger 1 so as to be electrically charged in a wireless manner easily and safely.

The wireless charger 1 is selected based on a variety of wireless charging standards, such as Wireless Power Consortium (i.e., Qi standard), Power Matters Alliance (PMA), and Alliance for Wireless Power (A4WP).

With reference to FIGS. 1-10, the wireless charger 1 further includes at least one transmitting coil module 11 electrically connected with the power source, and the wireless charger 1 electrically charges various mobile devices or terminal devices in the wireless manner.

Each of the at least one transmitting coil module 11 has a power circuit 13, a high-frequency oscillating circuit 15, and a high-frequency power amplifier circuit 17, wherein the power circuit 13 is electrically connected with the high-frequency oscillating circuit 15 and the high-frequency power amplifier circuit 17 so as to excite and transmit the electromagnetic waves. The wireless charging receiver 33 of the aroma diffuser 3 includes a receiving coil module 331 which has a high-frequency rectification filter circuit 333 configured to auxiliarily receive rectification voltage of the electric currents. In addition, the high-frequency rectification filter circuit 333 is electrically connected with the air conveyor 35.

Each transmitting coil module 11 further has a magnetic conductor and a transmitting control circuit. The receiving coil module 331 has a receiving control circuit or a rectification regulator circuit, and each transmitting coil module 11 produces the magnetic field which couples with the receiving coil module 331, such that the receiving coil module 331 is connected with magnetic field signals, and the receiving control circuit of the receiving coil module 331 converts the magnetic field signals into a power of the electricity applicable for the air conveyor 35.

Referring to FIGS. 1 and 4-6, the aroma diffuser 3 further includes a cap 304 which has an air outlet 305, the scatter path 306, and an arcuate face 307, wherein the air outlet 305 is defined on a top of the cap 304, the scatter path 306 is defined on a hollow space of an wall of the cap 304 and communicates with the air outlet 305, and the arcuate face 307 is arranged on the top of the cap 304 surrounding the air outlet 305, wherein a bottom of the cap 304 is movably connected with the opening 310 of the air guide holder 31. When the scent bottle 5 is accommodated into the second groove 314 of the air guide holder 31, the at least one part of the diffusing element 55 is located in a scatter path 306 of the cap 304, and the gap is formed between the at least one part of the diffusing element 55 and the air outlet 305 so that the external air flows through the first groove 312, the opening 310, and the scatter path 306 from the air conveyor 35. Thereafter, the external air flows through the gap between the diffusing element 55 and the air outlet 305 via the scatter path 306 by using the arcuate face 307 to diffuse the scent, hence the external air contacts with the diffusing element 55 by ways of the cap 304 to diffuse the scent strongly. Preferably, the aromatic liquid 51 does not contain volatile agent or has low volatility but diffuses the scent strongly.

As shown in FIGS. 7-9, when the scent carrier 5 is accommodated into the second groove 314 of the air guide holder 31, the scent carrier 5 is located in the scatter path 306 of the first groove 312, and a gap is formed between the scent carrier 5 and the air outlet 305 so that the external air flows through the scent carrier 5 and the opening 310 from the air conveyor 35. Thereafter, the external air flows toward the gap between the scent carrier 5 and the air outlet 305 via the scatter path 306 by using the arcuate face 307, thus diffusing the scent. Thereby, the external air contacts with the scent carrier 5 by ways of the cap 304 to diffuse the scent strongly. Preferably, the aromatic liquid 51 does not contain volatile agent or has low volatility but diffuses the scent strongly.

As illustrated in FIGS. 1 and 6-9, the first aperture 3160 is defined on an end of the guiding cavity 316 in an S-axis direction so that the external air flows into the second aperture 3161, the guiding cavity 316, and the first aperture 3160 from the air conveyor 35. Thereafter, the external air contacts with an inner fence 3121 of the first groove 312, the diffusing element 55 or the scent carrier 5, thus diffusing the scent effectively.

With reference to FIGS. 1 and 4-9, the air guide holder 31 and the guiding cavity 316 are cylindrical, the first aperture 3160 and the second aperture 3161 are circular, such that the external air flows through the second aperture 3161, the guiding cavity 316, the first groove 312, the opening 310, and the first aperture 3160 to surround the diffusing element 55 or the scent carrier 5, thus diffusing the scent effectively.

Referring to FIGS. 1 and 6-9, the first aperture 3160 is a porous structure so that a flow of the external air flowing through the first aperture 3160 is less than that flowing into the guiding cavity 316 from the second aperture 3161, and the guiding cavity 316 stores an air pressure so that the external air sprays out of the first aperture 3160 to contact with the diffusing element 55 or the scent carrier 5, thus diffusing the scent strongly.

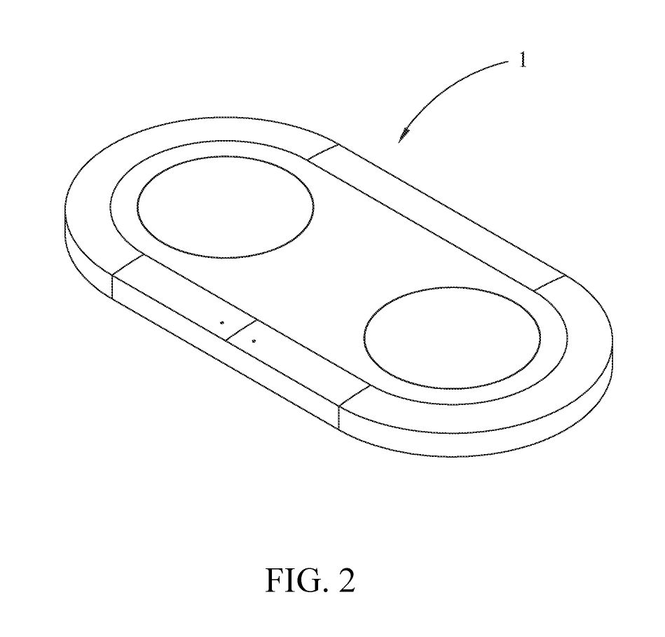

With reference to FIGS. 2, 3, 10 and 13, the wireless charger 1 of the scent diffusing system and device includes a charge board 17 which has a first lid 171 and a second lid 172 connected with the first lid 172, wherein the at least one transmitting coil module 11 of the wireless charger 1 is fixed in the charge board 17. In this embodiment, two transmitting coil modules 11 are provided. In another embodiment, multiple transmitting coil modules 11 are provided, and the charge board 17 of the wireless charger 1 is electrically connected with the power source and the multiple transmitting coil modules 11 so that the scent diffusing system and device are electrically charged by using the multiple transmitting coil modules 11 or/and electrically charges the various mobile devices or terminal devices in the wireless manner.

As illustrated in FIGS. 1-3, the wireless charger 1 of the scent diffusing system and device includes multiple transmitting coil modules 11 which are separately arranged in the charge board 17 and are parallelly connected so as to electrically charge mobile devices or terminal devices in the wireless manner and to heat the aromatic element 5. When the heater 3 is electrically charged, the multiple transmitting coil modules 11 electrically charge mobile phones, wireless mice, or calculators in the wireless manner.

Referring to FIGS. 1, 6, 8 and 9, the aroma diffuser 3 of the scent diffusing system and device further includes a built-in power interface 39 formed on the side of the case 30 and electrically connected with the air conveyor 35. The aroma diffuser 3 is electrically connected with a power cable and the built-in power interface 39 so as to be electrically charged in a wired manner.

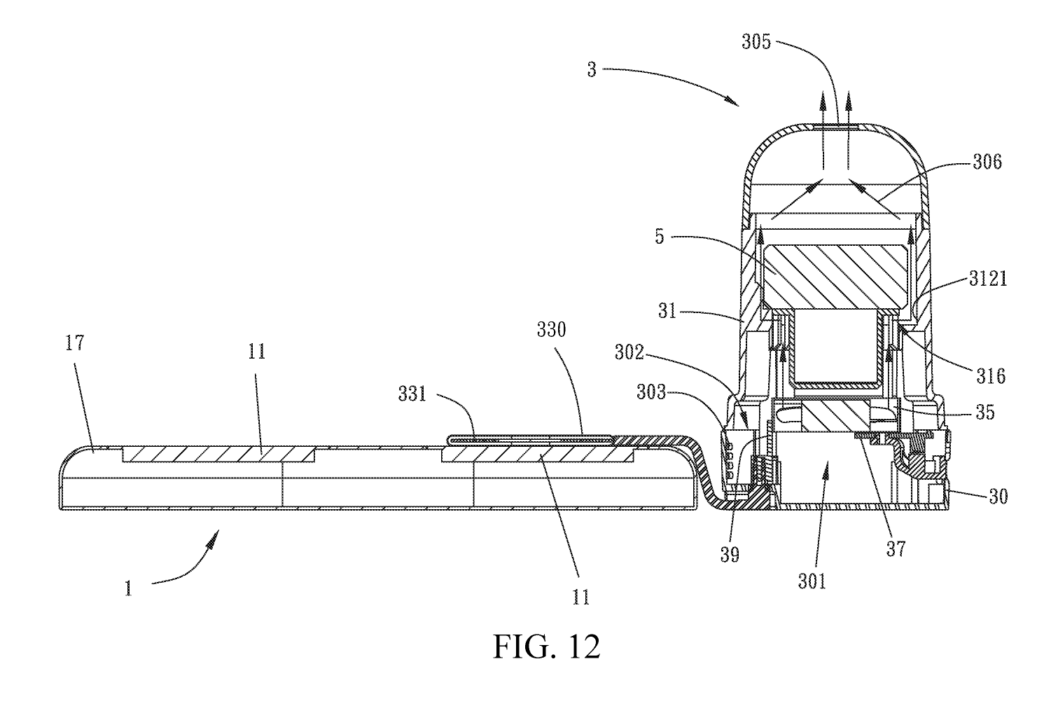

As shown in FIGS. 1, 5-9, 11, and 12, the aroma diffuser 3 of the scent diffusing system and device further includes a main control circuit 37 received in the receiving chamber 301 and configured to electrically connect with the air conveyor 35, the wireless charging receiver 33, and the built-in power interface 39 so as to turn on/off the scent diffusing system and device and the air conveyor 35.

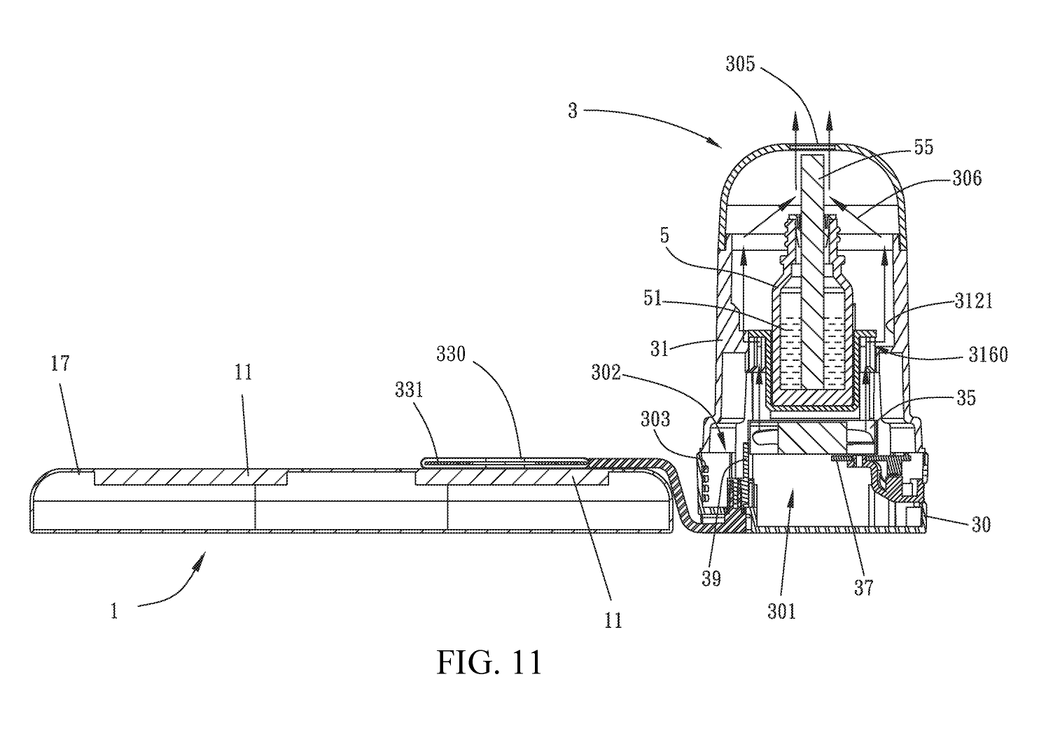

As illustrated in FIGS. 11-13, in another embodiment, an external scent diffusing device electrically charged in a wireless manner comprises an aroma diffuser 3 which is electrically wireless connected outside the wireless charger 1 and is electrically charged in the wireless manner by using the wireless charging receiver 33 (i.e., an externally wireless charging receiver 330), wherein the aroma diffuser 3 includes a built-in power interface 39 formed on the side of the case 30 and electrically connected with the air conveyor 35, a first side of the externally wireless charging receiver 330 is connected with the at least one transmitting coil module 11 of the wireless charger 1, and a second side of the externally wireless charging receiver 330 is connected with the built-in power interface 39 by ways of an electrical wire, such that the aroma diffuser 3 is electrically charged by using the externally wireless charging receiver 330 in the wired manner.

The wireless charger 1 is selected based on the variety of wireless charging standards, such as the Wireless Power Consortium (i.e., Qi standard), the Power Matters Alliance (PMA), and the Alliance for Wireless Power (A4WP). The externally wireless charging receiver 33 is selected based on the variety of wireless charging standards, such as the Wireless Power Consortium (i.e., Qi standard), the Power Matters Alliance (PMA), and the Alliance for Wireless Power (A4WP).

Referring to FIGS. 5-8, the aroma diffuser 3 is electrically charged in the wireless manner by using the externally wireless charging receiver 330, and the aroma diffuser 3 includes a case 30, an air guide holder 31, the externally wireless charging receiver 330, an air conveyor 35, a main control circuit 37, and a built-in power interface 39. The case 30 has an open segment 302 and a receiving chamber 301 defined in the open segment 302. The air conveyor 35 is accommodated is a bottom of the air guide holder 31, and the air guide holder 31 is connected above the open segment 302 of the case 30. The built-in power interface 39 is formed on a side of the receiving chamber 301, and the main control circuit 37 is electrically connected with the air conveyor 35 and a first side of the built-in power interface 39. The externally wireless charging receiver 330 is electrically connected with a second side of the built-in power interface 39 by using an electrical wire, wherein the externally wireless charging receiver 330 is close to the electromagnetic signals of the wireless charger 1 so as to induce the electromagnetic signals.

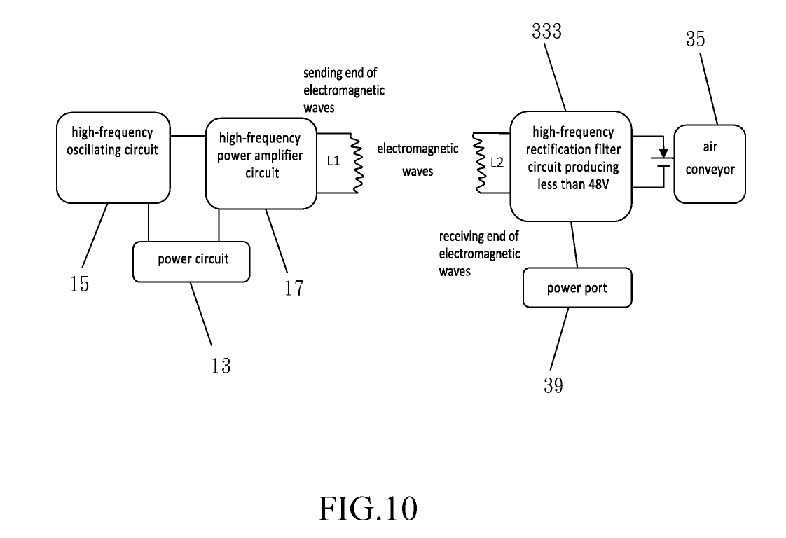

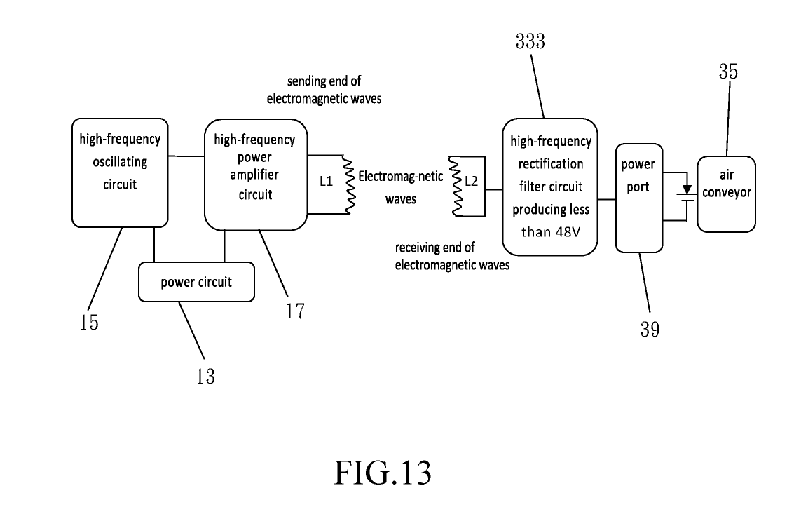

As shown in FIGS. 10 and 13, the aroma diffuser 3 further includes a receiving coil module 331 which has a high-frequency rectification filter circuit 333 configured to auxiliarily receive rectification voltage of the electric currents, the receiving coil module 331 is connected with the second side of the built-in power interface 39 by using another electrical wire.

While the preferred embodiments of the invention have been set forth for the purpose of disclosure, modifications of the disclosed embodiments of the invention as well as other embodiments thereof may occur to those skilled in the art. The scope of the claims should not be limited by the preferred embodiments set forth in the examples, but should be given the broadest interpretation consistent with the description as a whole.

What is claimed is:

1. A scent diffusing system electrically charged in a wireless manner comprising:
a wireless charger;
an aroma diffuser including a case, an air guide holder, a wireless charging receiver, and an air conveyor;
wherein the case has an open segment and a receiving chamber defined in the open segment, the air conveyor is accommodated in a bottom of the air guide holder, and the air guide holder is connected above the open segment of the case, the wireless charging receiver is accommodated in the receiving chamber, and the air conveyor is electrically connected with the wireless charging receiver;
wherein the aromatic element is received in the air guide holder of the aroma diffuser.

2. The scent diffusing system as claimed in claim 1, wherein the air guide holder has an opening, a first groove, a second groove, a guiding cavity, and a third groove, wherein the opening is defined on an upper end of the air guide holder, and the guiding cavity is defined between two inner walls of the air guide holder, the first groove extends to the air guide holder from the opening and communicates with an upper end of the guiding cavity, and the second groove is defined in a lower end of the guiding cavity, wherein an orifice of the second groove communicates with a bottom of the first groove; the guiding cavity has a first aperture formed on the upper end thereof and communicating with the first grove, the guiding cavity further has a second aperture formed on the lower end thereof and communicating with the receiving chamber; and the air conveyor is fixed in the third groove.

3. The scent diffusing system as claimed in claim 2, wherein the aroma diffuser further includes a cap which has an air outlet, the scatter path, and an arcuate face, wherein the air outlet is defined on a top of the cap, the scatter path is defined on a hollow space of an wall of the cap and communicates with the air outlet, and the arcuate face is arranged on the top of the cap surrounding the air outlet, wherein a bottom of the cap is movably connected with the opening of the air guide holder.

4. The scent diffusing system as claimed in claim 3, wherein the wireless charger further includes at least one transmitting coil module, and each of the at least one transmitting coil module has a power circuit, a high-frequency oscillating circuit, and a high-frequency power amplifier circuit, wherein the power circuit is electrically connected with the high-frequency oscillating circuit and the high-frequency power amplifier circuit; the wireless charging receiver of the aroma diffuser includes a receiving coil module which has a high-frequency rectification filter circuit which is electrically connected with the air conveyor.

5. The scent diffusing system as claimed in claim 4, wherein the wireless charger further includes multiple transmitting coil modules electrically connected therewith.

6. The scent diffusing system as claimed in claim 1, wherein the aroma diffuser further includes a main control circuit received in the receiving chamber and configured to electrically connect with the air conveyor, the wireless charging receiver, and the built-in power interface.

7. A scent diffusing device electrically charged in a wireless manner comprising:
an aroma diffuser including a case, an air guide holder, a wireless charging receiver, and an air conveyor;
wherein the case has an open segment and a receiving chamber defined in the open segment, the air conveyor is accommodated in a bottom of the air guide holder, and the air guide holder is connected above the open segment of the case, the wireless charging receiver is accommodated in the receiving chamber, and the air conveyor is electrically connected with the wireless charging receiver;
wherein the air guide holder has an opening, a first groove, a second groove, a guiding cavity, and a third groove, wherein the opening is defined on an upper end of the air guide holder, and the guiding cavity is defined between two inner walls of the air guide holder, the first groove extends to the air guide holder from the opening and communicates with an upper end of the guiding cavity, and the second groove is defined in a lower end of the guiding cavity, wherein an orifice of the second groove communicates with a bottom of the first groove; the guiding cavity has a first aperture formed on the upper end thereof and communicating with the first grove, the guiding cavity further has a second aperture formed on the lower end thereof and communicating with the receiving chamber; and the air conveyor is fixed in the third groove.

8. The scent diffusing device as claimed in claim 7, wherein the aroma diffuser further includes a cap which has an air outlet, the scatter path, and an arcuate face, wherein the air outlet is defined on a top of the cap, the scatter path is defined on a hollow space of an wall of the cap and communicates with the air outlet, and the arcuate face is arranged on the top of the cap surrounding the air outlet, wherein a bottom of the cap is movably connected with the opening of the air guide holder.

9. The scent diffusing device as claimed in claim 8 further comprising a wireless charger which includes at least one transmitting coil module, wherein each of the at least one transmitting coil module has a power circuit, a high-frequency oscillating circuit, and a high-frequency power amplifier circuit, wherein the power circuit is electrically connected with the high-frequency oscillating circuit and the high-frequency power amplifier circuit; the wireless charging receiver of the aroma diffuser includes a receiving coil module which has a high-frequency rectification filter circuit which is electrically connected with the air conveyor.

10. The scent diffusing device as claimed in claim 9, wherein the wireless charger includes a charge board which has a first lid and a second lid connected with the first lid, wherein the at least one transmitting coil module of the wireless charger is fixed in the charge board.

11. The scent diffusing device as claimed in claim 10, wherein the wireless charger further includes multiple transmitting coil modules which are separately arranged in the charge board and are electrically connected with the wireless charger.

12. The scent diffuser device as claimed in claim 11, wherein the aroma diffuser further includes a built-in power interface formed on a side of the case and electrically connected with the air conveyor.

13. The scent diffusing device as claimed in claim 7, wherein the aroma diffuser further includes a main control circuit received in the receiving chamber and configured to electrically connect with the air conveyor, the wireless charging receiver, and the built-in power interface.

14. The scent diffusing device as claimed in claim 9, wherein the aroma diffuser further includes a main control circuit received in the receiving chamber and configured to electrically connect with the air conveyor, the wireless charging receiver, and the built-in power interface.

15. An external scent diffusing device electrically charged in a wireless manner comprising:
an aroma diffuser which is electrically wireless connected outside the wireless charger and is electrically charged in the wireless manner by using an externally wireless charging receiver;
wherein the aroma diffuser includes a case, an air guide holder, the externally wireless charging receiver, an air conveyor, a main control circuit, and a built-in power interface;
wherein the case has an open segment and a receiving chamber defined in the open segment, the air conveyor is accommodated is a bottom of the air guide holder, and the air guide holder is connected above the open segment of the case;
wherein the built-in power interface is formed on a side of the receiving chamber, and the main control circuit is electrically connected with the air conveyor and a first side of the built-in power interface;
wherein the externally wireless charging receiver is electrically connected with a second side of the built-in power interface by using an electrical wire.

16. The external scent diffusing device as claimed in claim 15, wherein the air guide holder has an opening, a first groove, a second groove, a guiding cavity, and a third groove, wherein the opening is defined on an upper end of the air guide holder, and the guiding cavity is defined between two inner walls of the air guide holder, the first groove extends to the air guide holder from the opening and communicates with an upper end of the guiding cavity, and the second groove is defined in a lower end of the guiding cavity, wherein an orifice of the second groove communicates with a bottom of the first groove; the guiding cavity has a first aperture formed on the upper end thereof and communicating with the first grove, the guiding cavity further has a second aperture formed on the lower end thereof and communicating with the receiving chamber; and the air conveyor is fixed in the third groove.
